(19) United States
(12) Patent Application Publication
(10) Pub. No.: US 2005/0153352 A1
(43) Pub. Date: Jul. 14, 2005

(54) Cancer specific gene MG20

(76) Inventors: Stanley Chang, Gueishan Township (TW); Hsun-Lang Chang, Gueishan Township (TW); Wei-Ying Kuo, Gueishan Township (TW); Kuo-Yen Chen, Gueishan Township (TW); Ning-Yi Li, Gueishan Township (TW)

Correspondence Address: DINSMORE & SHOHL LLP;One Dayton Centre, Suite 1300, One South Main Street, Dayton, OH, 45402-2023, US

(21) Appl. No.: 11/031,922

(22) Filed: Jan. 7, 2005

Related U.S. Application Data
(60) Provisional application No. 60/535,102, filed on Jan. 9, 2004.

(51) Int. Cl. C12Q 1/68; G01N 33/574; C07H 21/04; C07K 16/30

(52) U.S. Cl. 435/6; 435/7.23; 530/388.8; 536/24.3

(57) ABSTRACT

MG20 (CCNDBP1) is identified as an oncogene. Methods and compositions for detecting and diagnosing cancer in patients are provided, by determining the level of MG20 expression in biological samples. Also provided are methods for screening for inhibitors and moderators of MG20 expression and activity, as well as compositions comprising compounds and molecules that inhibit or moderate MG20 expression or activity, thereby treating cancer, in vivo.

### CROSS-REFERENCE TO RELATED APPLICATIONS

This application claims the benefit of U.S. Provisional Application Ser. No. 60/535,102, filed Jan. 9, 2004.

### FIELD OF THE INVENTION

The present invention relates to detection, diagnosis and prevention of cancer in patients. The invention further relates to methods and compositions for treating cancer and cancer related diseases.

### BACKGROUND OF THE INVENTION

Cancer is the uncontrolled growth and spread of cells that may affect almost any tissue of the body. Lung, colorectal and stomach (gastric) cancer are among the five most common cancers in the world for both men and women.

In the developed world cancer is a leading cause of morbidity and mortality in adults. In the United States up to one third of the population develop cancer during their life span. As a result, in the USA alone it is estimated that about 500,000 individuals die from cancer annually (Ahmedin et al., CA Cancer J. Clin. 52:23-47 (2002)). With global improvements in public health provision, populations are increasingly living to a greater age. Cancer has often been termed a ‘disease of the old’ and, as such, prevalence of cancer as a primary cause of mortality and morbidity is projected to increase in the years to come.

Turning to the biological perspective, as a normal cell progresses through the complex process of transformation to reach a cancerous, or ‘neoplastic’, state, its gene expression profile can typically change. Expression of certain genes that are usually dormant in a normal healthy cell may be turned on or up regulated in a cancer cell. Likewise, genes that maintain a normal healthy phenotype can be switched off or suppressed in cancer cells. Consequently, such genes can be viewed as biomarkers of neoplastic behavior in cells. In some circumstances the cell itself might not yet have even transformed into a cancerous cell, but may only be predisposed to such a change in future.

Previously, identification of cancers—for example gastric cancer—has relied upon traditional methods of diagnosis. For instance, detection of tumors in the stomach and esophagus is usually achieved by radiography, following ingestion of a ‘barium meal’. This results in potentially harmful exposure of the patient to X-rays. Hence, a less invasive method of tumor diagnosis, such as via blood or urine analysis is desirable.

Public awareness campaigns alerting individuals to the possible warning signs of cancer can have an impact on diagnosis and treatment of the disease. Large scale cancer screening of otherwise healthy populations has also proven successful in some instances. Screening refers to the use of simple tests across a healthy population in order to identify individuals who have disease, but do not yet have symptoms. Examples include breast cancer screening using mammography and cervical cancer screening using cytology screening methods, including Pap smears. In order for screening to be successful it must be relatively simple to perform and consistently reliable.

However, there are currently very few large-scale screening programmes for cancers other than breast and cervical cancers. As a result, in many cases the disease can remain symptomatically undetectable in the patient until a very advanced stage, such as when the patient actually experiences pain. Hence, there is a need to identify novel markers of the presence of cancer and also the progression of the disease once it has become established in a patient. In particular, there is a need to identify novel markers of the cancer that can be suitable candidates for inclusion in large scale screening programmes. In addition, there is a need to identify genes that are involved in the development and progression of cancer so that therapies can be designed accordingly.

In the present invention a novel marker for cancer called MG20 has been identified. MG20 corresponds to Cyclin D-type binding-protein 1 (CCNDBP1), a 232 amino acid residue polypeptide, see Terai et al. (Hepatology 32 (2), 357-366 (2000)), Yamada et al. (Hepatology 32 (2), 278-288 (2000)), Xia et al. (J. Biol. Chem. 275 (27), 20942-20948 (2000), and Yao et al. (Exp. Cell Res. 257 (1), 22-32 (2000)).

The present inventors have shown that expression of the MG20 gene is detectable in the blood of patients and the level of expression of the gene is correlated to the presence of cancer. According to the invention, MG20 is further identified as an oncogene, elevated expression of which is capable of inducing transformation in mammalian cells, and inhibition of which can suppress malignancy in cancer cells. The invention also provides methods and compositions for treating cancer, in particular ovarian, thyroid, testis, uterine, prostate, kidney and gastric cancers, in patients.

These and other uses, features and advantages of the invention should be apparent to those skilled in the art from the teachings provided herein.

### SUMMARY OF THE INVENTION

In accordance with a first aspect of the invention there is provided a method of detecting and diagnosing cancer in a patient in need thereof, comprising the steps of obtaining a biological sample from said patient and analysing said biological sample for expression of MG20. The step of detecting a cancer in a patient can, if appropriate, include the step of diagnosing the patient as having cancer. In one embodiment, expression of MG20 at a level in excess of a normal level is indicative of the presence of neoplastic tissue, such as cancer or other tumors. In a further embodiment, the biological sample comprises cells obtained from a biological source selected from: tissues; whole blood; serum; plasma; saliva; cerebrospinal fluid; ascites fluid; pleural fluid and urine. Alternatively, the cells may obtained from a biopsy of a suspected tumor. In a specific embodiment the biological sample comprises peripheral blood mononuclear cells (PBMCs).

In a second aspect of the invention, a method is characterised by the steps of:
(a) providing a biological sample from a subject;
(b) detecting the level of MG20 in the biological sample; and
(c) comparing the level of MG20, in the biological sample with that in a control sample obtained from a healthy individual;
wherein an increased level of MG20, in the biological sample compared to that in the control sample indicates the presence of neoplasm in the subject. The level of MG20 can be the polynucleotide level or a portion, fragment, variant or complementary strand thereof, mRNA level, cDNA level, polypeptide level or a portion or fragment thereof, protein level or a level of biological activity of MG20.

Third and fourth aspects of the invention provide for diagnostic kits in which either comprise at least one nucleic acid probe that consists of a nucleotide sequence that is capable of hybridizing under conditions of medium stringency with about 15 contiguous bases of SEQ ID NO:1; or which comprise at least one antibody, said antibody being capable of specifically binding to an MG20 polypeptide.

A fifth aspect of the invention provides for an in vitro method for monitoring the progression of cancer in a patient, comprising the steps of:
(a) contacting a biological sample from the patient with a substance selected from polynucleotides, probes, primers, or a portion, fragment, variant or complementary strand thereof; or polypeptides, antibodies that interact with MG20, or a portion or fragment thereof;
(b) detecting the level of MG20 polynucleotide or polypeptide, or the portion, fragment, variant or complementary strand thereof, that interacts with the substance in the sample;
(c) repeating steps (a) and (b) using a biological sample from the patient at a subsequent point of time; and
(d) comparing the level detected in step (c) with that in step (b) and therefrom monitoring the progression of the cancer in the patient.

In a preferred embodiment of the invention the method further comprises the steps of obtaining a plurality of biological samples from said patient at a plurality of time intervals in a time course, and comparing the expression of MG20 in each biological sample, thereby effecting a diagnosis of cancer progression in said patient over said time course. The monitoring of a cancer's progression allows for a more accurate determination of the prognosis of a given cancer patient.

In a further aspect of the invention the method of the fourth aspect can be employed for monitoring patients for the recurrence of cancer, for example after a period of remission. A related aspect of the invention provides for a method of the fourth aspect of the invention for determining the prognosis of cancer in a patient. In this aspect, determination of MG20 expression levels allows the determination of the progress and likely outcome of cancer and cancer related diseases in a patient suffering from cancer.

A yet a further aspect of the invention provides for a polynucleotide vector comprising an isolated nucleic acid sequence that is substantially complimentary to at least 18 contiguous nucleotides of the nucleic acid molecule of SEQ ID NO:1, a transcription promoter, and a transcription terminator, wherein the promoter is operably linked to the nucleic acid sequence that is substantially complimentary to a nucleic acid molecule of SEQ ID NO:1, and wherein the isolated nucleic acid sequence that is substantially complimentary to a nucleic acid molecule of SEQ ID NO:1 is operably linked with the transcription terminator. The vector of the invention is preferably an expression vector or a vector that is capable of generating short interfering double stranded RNAs (RNAi) in a transfected cell.

Further aspects of the invention provide for cells, that comprise a vector of the invention. Recombinant host cells comprising the polynucleotide vector of the invention are provided, wherein the host cell is suitably selected from a bacterium; yeast; fungal cells; insect cells; mammalian cells; and plant cells. An aspect of the invention further provides a method of producing an MG20 polypeptide, the method comprising culturing the aforementioned recombinant host cells that comprise an expression vector of the invention, and that produce said polypeptide, and then isolating the polypeptide.

Another aspect of the invention, there is provided an antibody or antibody fragment that specifically binds to a polypeptide of SEQ ID NO: 2. In preferred embodiments the antibody is selected from a polyclonal antibody; a murine monoclonal antibody; a humanized monoclonal antibody derived from a murine monoclonal antibody; a human monoclonal antibody; and a fab antibody fragment. Another aspect of the invention provides for an anti-idiotypic antibody that specifically binds to an antibody or antibody fragment of invention.

An additional aspect of the invention provides a method for inhibiting malignancy in cancer cells, the method comprising exposing said cancer cells to an inhibitor of MG20. In specific embodiments of the invention the inhibitor of MG20 comprises a moiety selected from the group consisting of: a polynucleotide sequence that is substantially complimentary to the sequence of SEQ ID NO: 1; an oligonucleotide sequence that is substantially complimentary to at least 12 contiguous bases of SEQ ID NO:1; an oligonucleotide RNAi sequence that is substantially complimentary to at least 18 contiguous bases of SEQ ID NO:1; an antibody of claim 24; a small molecule; a glycoprotein; and a polysaccharide.

Further aspects of the invention provide pharmaceutical compositions for the prevention, treatment and/or therapy of cancer in a patient. In a first instance the pharmaceutical composition comprises:
an inhibitor of MG20 selected from the group consisting of: a polynucleotide sequence that is substantially complimentary to the sequence of SEQ ID NO: 1; an oligonucleotide sequence that is substantially complimentary to at least 12 contiguous bases of SEQ ID NO:1; an oligonucleotide RNAi sequence that is substantially complimentary to at least 18 contiguous bases of SEQ ID NO:1; an expression vector construct comprising said polynucleotide sequence; an expression vector construction comprising said oligonucleotide sequence; an antibody as described previously; a small molecule; a glycoprotein; and a polysaccharide; and
a pharmaceutically acceptable excipient and carrier.

Whereas, in a second instance of this aspect of the invention the pharmaceutical composition comprises:
a polypeptide with the sequence set forth in SEQ ID NO:2, or a portion or fragment thereof; and
a pharmaceutically acceptable excipient and carrier.

Methods for the treatment of patients in need thereof comprising administration of the pharmaceutical compositions of the invention are also provided in further aspects of the invention.

A still further aspect of this invention provides for vaccine compositions, comprising a polypeptide of SEQ ID NO:2, or an antigenic fragment of said polypeptide, and a pharmaceutically acceptable carrier. In another aspect of the invention the vaccine composition comprises a polynucleotide vector of the invention, and a pharmaceutically acceptable carrier. For both aspects, a specific embodiment of the invention provides for the additional inclusion of a non-specific immune response adjuvant in the vaccine composition.

Another aspect of the invention provides for a method for identifying a molecule that interacts with MG20 comprising:
a) screening a plurality of candidate molecules in order to identify one or more target molecules that bind to MG20 polypeptide;
b) determining whether said one or more target molecules interacts with MG20 polypeptide so as to moderate MG20 biological activity; and
characterising a target molecule that moderates MG20 biological activity as an MG20 interacting molecule.

A further aspect of the invention provides a method for identifying a molecule that moderates the expression of MG20 in a cell comprising:
a) exposing a cell that expresses MG20 to a candidate molecule in order to identify whether said candidate molecule has a moderating effect on the expression of MG20 in the cell;
b) determining whether said candidate molecule moderates MG20 expression levels; and
c) characterising a candidate molecule that selectively moderates MG20 expression levels in the cell as an MG20 moderator molecule.

These and other features, aspects and advantages of the present invention will become better understood with regard to the following description, appended claims and accompanying drawings that are provided only for further elaboration without limiting or restricting the present invention, where:

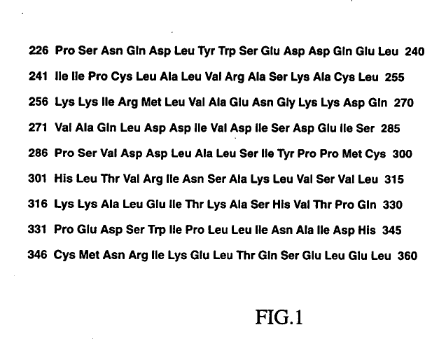

FIG. 1 shows both the full length DNA (SEQ ID NO: 1) and deduced protein sequence (SEQ ID NO: 2) of MG20.

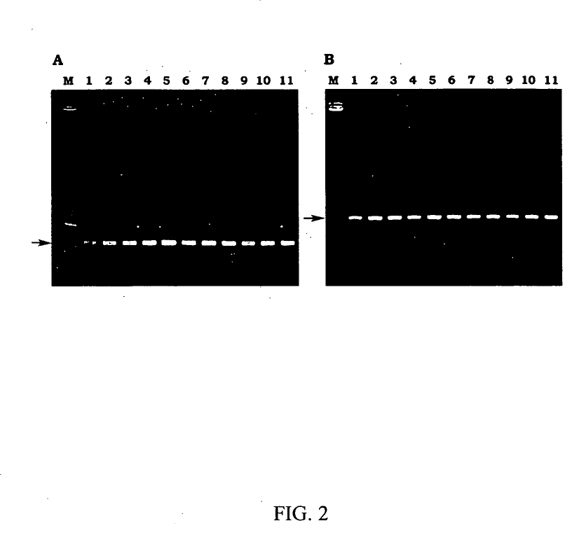

FIG. 2A shows the RT-PCR result of MG20 expression level in the peripheral blood mononuclear cell (PBMC) sample and cancer cell lines.

FIG. 2B shows the RT-PCR result of GAPDH expression level in the PBMC sample and cancer cell lines.

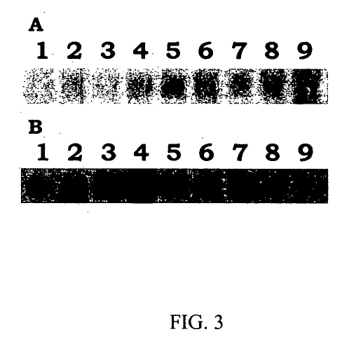

FIG. 3A shows the Northern blot result of MG20 expression level in the PBMC sample and cancer cell lines.

FIG. 3B shows the Northern blot result of β-actin expression level in the PBMC sample and cancer cell lines.

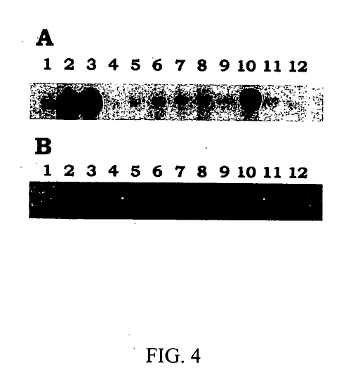

FIG. 4A shows the Northern blot result of MG20 expression level in 12 different human normal, non-cancerous tissues.
FIG. 4B shows the Northern blot result of β-actin expression level in 12 different human normal, non-cancerous tissues.

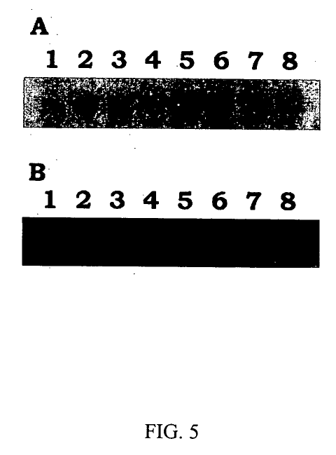

FIG. 5A shows the Northern blot result of MG20 expression level in 8 different human tumor tissues.

FIG. 5B shows the Northern blot result of β-actin expression level in 8 different human tumor tissues.

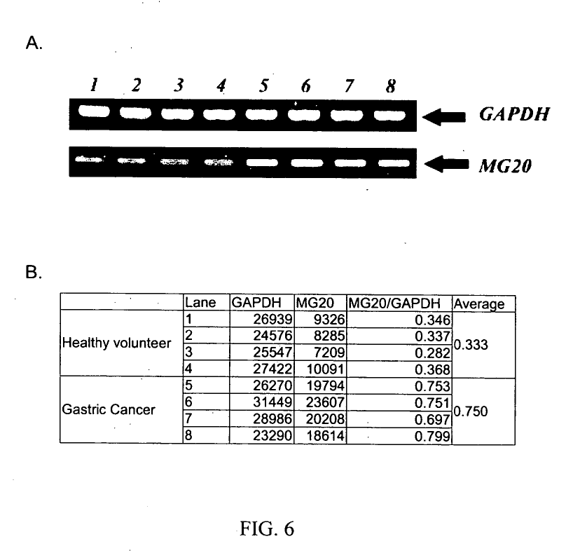

FIG. 6A shows the RT-PCR result of MG20 expression levels in the PBMC samples from normal individuals and gastric cancer patients.

FIG. 6B shows the quantitative overexpression levels of MG20 in gastric cancer patients.

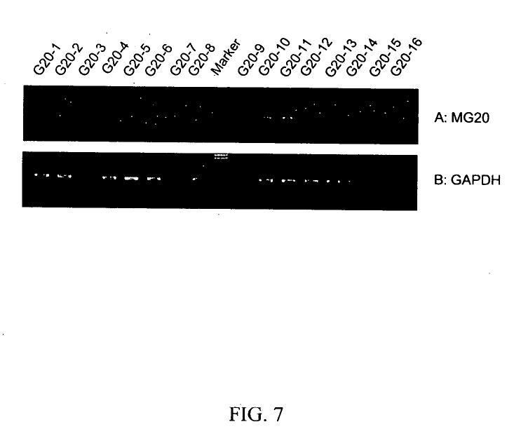

FIG. 7A shows the RT-PCR result of MG20 expression level in MG20-transfected NIH 3T3 cells.

FIG. 7B shows the RT-PCR result of GAPDH expression level in MG20-transfected NIH 3T3 cells.

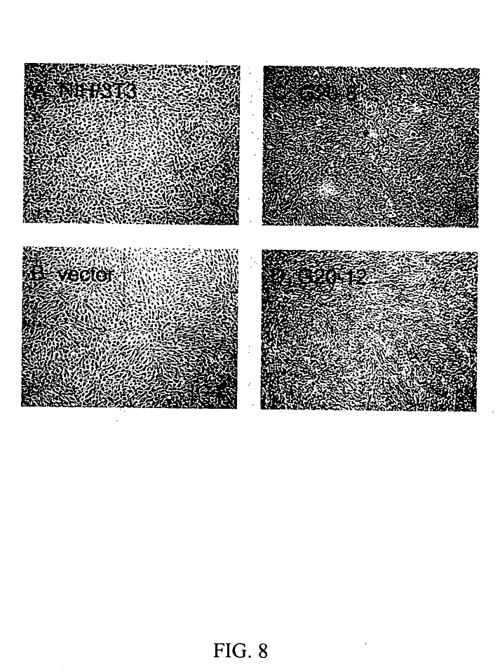

FIG. 8. shows the morphological transformation caused by MG20 overexpression in NIH 3T3 cells.

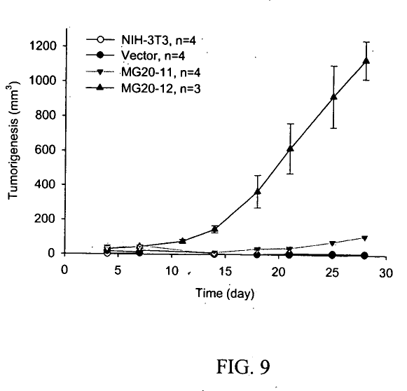

FIG. 9 shows the tumor development induced by MG20 overexpression in NIH 3T3 cells.

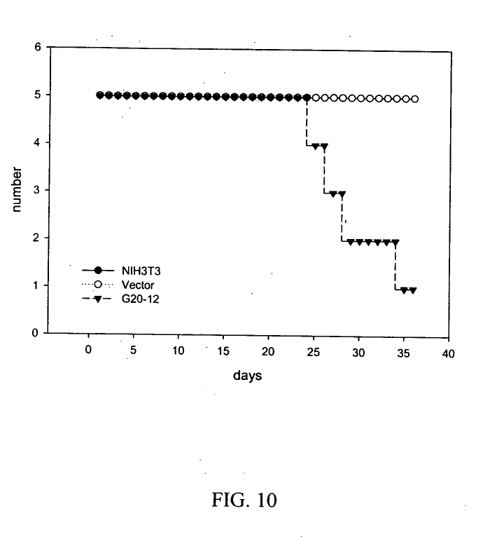

FIG. 10 shows the metastasis induced by MG20 transfectants.

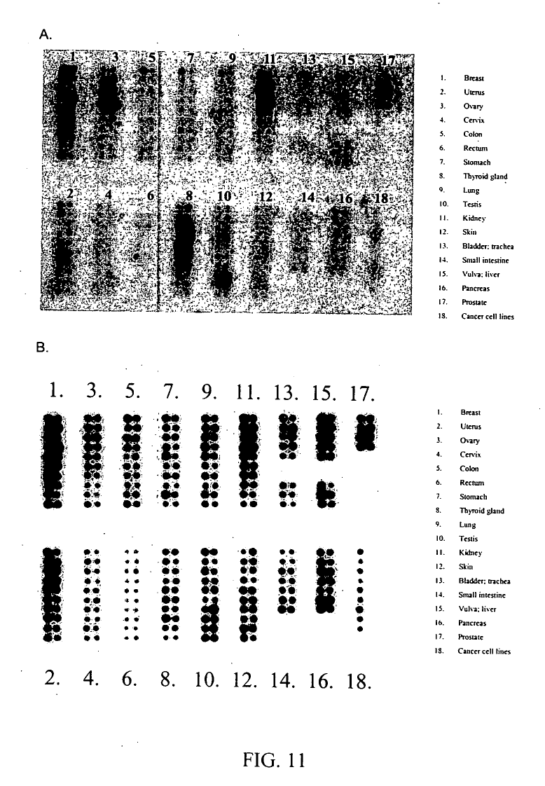

FIG. 11A shows the result of RNA profiling assay for MG20.

FIG. 11B shows the result of RNA profiling assay for ubiquitin.

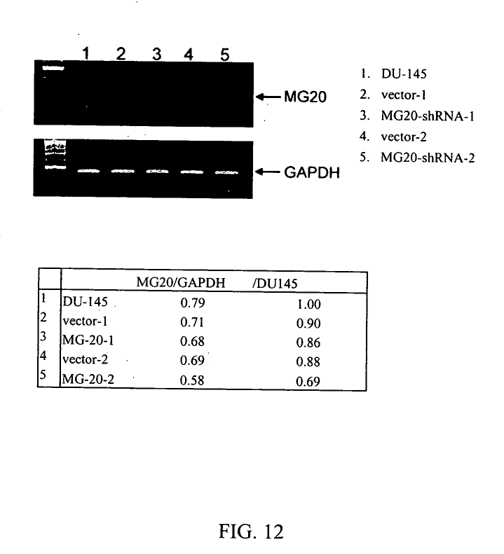

FIG. 12 shows the reduction of MG20 levels in DU145 prostate cancer cells transfected with MG20 targeting shRNA.

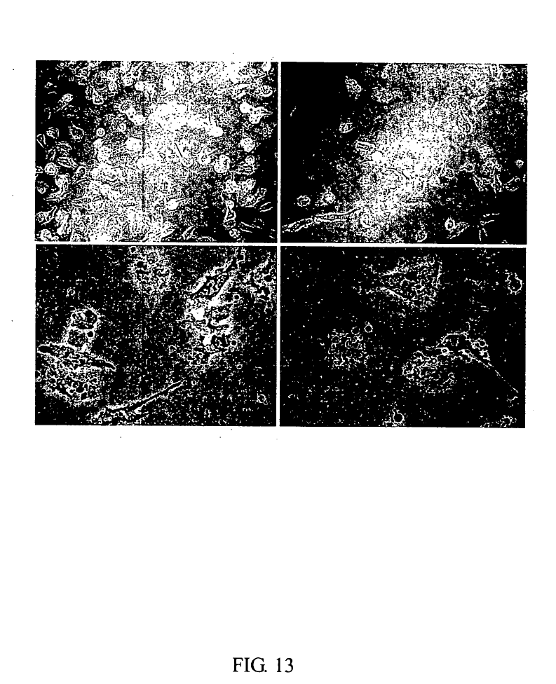

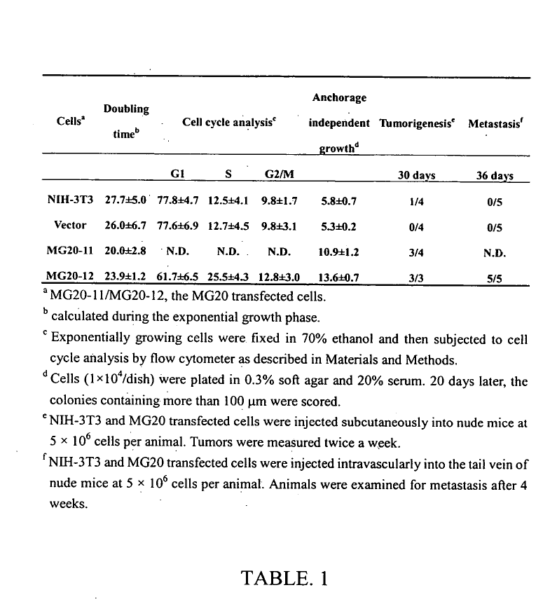

FIG. 13 shows the anti-malignancy effect of MG20 silencing by shRNA methodology in DU-145 prostate cancer cells.

TAB. 1 shows the transforming properties of MG20 transfected NIH 3T3 cells.

All references cited herein are incorporated by reference in their entirety. Unless otherwise defined, all technical and scientific terms used herein have the same meaning as commonly understood by one of ordinary skill in the art to which this invention belongs.

### DETAILED DESCRIPTION OF THE INVENTION

The present invention is based, in part, upon the discovery that the MG20 gene is expressed at elevated levels when neoplasms are present in a patient.

Elevated levels of MG20 expression in PBMCs are a diagnostic indicator of cancer in a patient. This correlation is of particular note in relation to the diagnosis of gastric cancer. The invention is of particular advantage as it allows for mass screening of populations for the presence of neoplastic tissue, such as ovarian, thyroid, testis, uterine, prostate, kidney and gastric cancer, by a simple blood test. The present invention, therefore, provides methods, apparatus and compositions for the diagnosis and treatment of cancers in patients.

As mentioned previously, the target gene of the present invention, MG20, corresponds to Cyclin D-type-binding protein 1 (CCNDBP1; Accession No: NM_{—}03737d). CCNDBP1 is a nuclear protein that interacts with the leukocyte-specific adaptor protein Grap2 and the key cell cycle protein, cyclin D (Xia et al., supra). The CCNDBP1 gene is mapped to reside on chromosome 15q14-q15, and encodes a deduced 360-amino acid protein with a calculated molecular mass of 40 kD. Structural prediction shows this protein contains a helix-loop-helix region without the basic DNA-binding domain functionally related to the ID (inhibitor of DNA binding) protein family, a dominant negative regulator for transcription factors. The ID protein family members have been implicated in various significant cellular functions, such as embryonic development, lymphopoiesis, cell cycle control and oncogenesis. Following the putative helix-loop-helix domain are a central acidic domain and a leucine zipper motif, suggesting that these regions may be involved in interactions with other proteins (Xia et al. supra). The interaction between CCNDBP1 and cyclin D is thought to influence the phosphorylation status of retinoblastoma protein (Rb), leading to inhibitory effects of E2F transcriptional activity. Given the roles of Rb and E2F in cell cycle progression and cell proliferation, CCNDBP1 may be involved in the cell cycle regulation pathway. CCNDBP1 was also known to interact with Grap2, which is a leukocyte-specific adaptor protein found primarily in T lymphocytes, monocytes/macrophages and reported important for immune cell signaling through interacting with different signaling molecules and forming signal transduction complexes. The interaction between CCNDBP1 and Grap2 is thought to facilitate the proliferation of T lymphocytes during their activation, implicating a possible role of CCNDBP1 concerning cell cycle regulation or cell proliferation in the immune system.

However, there has been no identified correlation between CCNDBP1 and cancer in the art. Until now CCNDBP1 was not a known oncogene. Indeed, the present invention is based upon the unexpected and surprising correlation identified by the inventors between the level of expression of MG20 (CCNDBP1) in circulating PBMCs and the presence of cancerous tissue in a patient, particularly gastric cancer. Hence, in one embodiment of the present invention MG20 represents a potent biomarker for cancer.

Although a number of potential biomarkers have been found for diagnosing presence of neoplasms in patients, the limited practical success of these biomarkers is still a problem either due to the low sensitivity or non-specificity of prior art biomarkers. Given the significance in the levels of differential expression of MG20 between neoplastic and normal cells as well as the high expression of MG20 in PBMCs, MG20 can be utilized as a biomarker for sensitive, non-invasive detection and diagnosis of tumors whilst still at an early stage in the disease.

The full length sequence of the MG20 gene (SEQ ID NO: 1) and the deduced amino acid sequence (SEQ ID NO: 2) are shown in FIG. 1, respectively. As noted above, the present invention is generally related to methods for the prevention, detection, treatment, monitoring, therapy and diagnosis of neoplasms. The methods may use MG20 polypeptides, polynucleotides encoding such polypeptides, compositions comprising the polypeptides and/or polynucleotides, functional fragments of the polypeptides, antigenic fragments of the polypeptides, antibodies, antibody fragments, antigen presenting cells and/or immune system cells.

In setting forth the invention, a number of definitions are provided that will assist in the understanding of the invention.

The term “neoplasm” used herein refers to any new and abnormal cellular growth, specifically a new growth of tissue or cells in which the growth is uncontrolled and progressive. In solid tissues neoplasms typically result in the formation of masses that are termed “tumors”. In the art, malignant neoplasms are typically referred to as “cancers”. A malignant neoplasm is distinguished from a benign one in that the former shows a greater degree of anaplasia and typically has the properties of invasiveness and metastasis. Invasiveness refers to the local spread of a neoplasm by infiltration or destruction of surrounding tissue, typically breaking through the basal laminas that define the boundaries of the tissues, thereby often entering the body's circulatory system. Metastasis typically refers to the dissemination of tumor cells by lymphatics or blood vessels. Metastasis also refers to the migration of tumor cells by direct extension through serous cavities, or subarachnoid or other spaces within the body. Through the process of metastasis, tumor cells can migrate and establish neoplasm in distant areas away from the site of initial appearance. Hence, it is apparent that many molecular interactions controlled by a series of genetic transformations can govern the transition of a normal cell to a neoplastic or cancerous state.

Accordingly, the present invention pertains to an isolated nucleic acid molecule comprising a mammalian (e.g., primate or human) MG20 gene. As used herein, the term “MG20” refers to an isolated nucleic acid molecule in the 15q14-q15 locus, which is associated with a susceptibility to neoplasm and the related syndromes or conditions. The present invention also relates to an isolated nucleic acid molecule (e.g., cDNA or the gene) that encodes a MG20 polypeptide (e.g., the polypeptide as shown in FIG. 1, or another variant of a MG20 polypeptide). More preferably, an isolated nucleic acid molecule encodes an immunogenic portion of MG20 polypeptide. In a preferred embodiment, the isolated nucleic acid molecule comprises SEQ ID NO: 1 (as shown in FIG. 1) or the complementary strand of SEQ ID NO: 1.

The isolated nucleic acid molecule of the invention can be RNA, for example, mRNA or HnRNA, or DNA, such as cDNA and genomic DNA. A “MG20 nucleic acid”, as used herein, refers to a nucleic acid molecule (RNA, mRNA, cDNA, or genomic DNA, either single- or double-stranded) encoding MG20. DNA molecules can be doubled-stranded or singled-stranded; single stranded RNA or DNA can be either coding, or sense, strand, or the non-coding, or antisense, strand. The nucleic acid molecule can include all or a portion of the coding sequence of the gene and can further comprise additional non-coding sequences such as introns and non-coding 3′ and 5′ sequences (including promoter, regulatory, poly-A stretches or enhancer sequences, for example). In addition, the nucleic acid molecule can be fused to another sequence, for example, a label, a marker or a sequence that encodes a polypeptide that assists in isolation or purification of the polypeptide. Such sequences include, but are not limited to, those that encode a selection marker (e.g. an antibiotic resistance gene, or a reporter sequence), those that encode a repetition of histidine (HIS tag) and those that encode a glutathione-S-transferase (GST) fusion protein.

The term “isolated”, when applied to a polynucleotide sequence herein, denotes that the sequence has been removed from its natural organism of origin and is, thus, free of extraneous or unwanted coding or regulatory sequences. An isolated nucleic acid molecule or nucleotide sequence can include a nucleic acid molecule or nucleotide sequence which is synthesized chemically or by recombinant means. The isolated sequence is suitable for use in recombinant DNA processes and within genetically engineered protein synthesis systems. Such isolated sequences include cDNAs and genomic clones. The isolated sequences may be limited to a protein encoding sequence only, or can also include 5′ and 3′ regulatory sequences such as promoters, enhancers and transcriptional terminators. If the isolated sequence is of genomic origin then it may also comprise non-coding regions, such as introns.

The nucleic acid molecules of the invention can be fused to other coding or regulatory sequences and still be considered as “isolated”. Thus, recombinant MG20 DNA contained in a vector is included in the definition of “isolated” as used herein. Also, isolated nucleic acid molecules include recombinant DNA molecules in heterologous cells, as well as partially or substantially purified DNA molecules in solution. “Isolated” nucleic acid molecules also encompass in vivo or in vitro RNA transcripts of MG20. Such isolated nucleotide sequences are useful in the manufacture of the encoded polypeptide, as probes for isolating homologous sequences (e.g., from other mammalian species), for gene mapping (e.g., by in situ hybridization with chromosome), or for detecting overexpression of a gene in tissues. For example, in accordance with the present invention MG20 expression can be detected in human blood tissues, more preferably in PBMCs, using isolated nucleic acid sequences as probes via a standard technique such as Northern blot analysis.

The present invention, thus, provides for diagnosis and analysis of cancer in biological samples containing elevated levels of expression of mRNA and polypeptide sequences that have substantially similar sequence identity or homology to that of SEQ ID NO: 1 or 2 respectively. The term “substantially similar sequence identity” is used herein to denote a level of sequence similarity of from about 50%, 60%, 70%, 80%, 90%, 95% to about 99% identity. Percent sequence identity can be determined using conventional methods (Henikoff and Henikoff Proc. Natl. Acad. Sci. USA 1992; 89:10915, and Altschul et al. Nucleic Acids Res. 1997; 25:3389-3402).

The level of similarity between two given nucleic acid sequences can be measured, to an extent, by testing the ability of complimentary strands of DNA to hybridize under certain conditions. Nucleic acid molecules which hybridize under high stringency hybridization conditions are said to be most similar and, thus, exhibit high levels of sequence similarity. The present invention includes nucleic acid molecules which hybridize under medium to high stringency hybridization conditions to a nucleotide sequence comprising a nucleotide sequence selected from SEQ ID NO: 1 or the complementary strand of SEQ ID NO: 1. “Stringency hybridization conditions” is a term of the art which refers to the incubation and wash conditions, e.g., conditions of temperature and buffer concentration, which permit hybridization of a particular nucleic acid to a second nucleic acid. For example, certain stringency hybridization conditions can be used which distinguish complementary nucleic acids from those of less complementarity. “High”, “moderate”, and “low” stringency hybridization conditions for nucleic acid hybridization are explained in Current Protocols in Molecular Biology (Ausubel, F. M. et al., John Wiley & Sons (1998)). For example, a low stringency wash can comprise washing in a solution containing 0.2×SSC/0.1.% SDS for 10 min at room temperature; a moderate stringency wash can comprise washing in a pre-warmed (42° C.) solution containing 0.2×SSC/0.1% SDS for 15 min at 42° C.; and a high stringency wash can comprise washing in a pre-warmed (48° C.) solution containing 0.1×SSC/0.1% SDS for 15 min at 48° C. Further, washes can be performed repeatedly or sequentially to obtain a desired result as known in the art.

The present invention also provides isolated nucleic acid molecules that contain a fragment, portion or variant that hybridizes under high stringency conditions to a nucleotide sequence comprising a nucleotide sequence selected from SEQ ID NO: 1 or the complementary strand of SEQ ID NO: 1. The nucleic acid fragments of the invention are at least about 15, preferably at least 18, 21, or 25 nucleotides, and can be 40, 50, 70, 100, 20.0, or more nucleotides in length. Longer fragments, for example, 30 or more nucleotides in length, which encode antigenic polypeptides described hereinafter, are particularly useful, such as for the generation of antibodies.

Particular small nucleic acid molecules that are of use in the invention are short stretches of double stranded RNA that are known as short interfering RNAs (siRNAs). These interfering RNA (RNAi) techniques allow for the selective inactivation of gene function in vivo. In the present invention, RNAi has been used to knock-down MG20 expression in a human ovarian cancer cell line and in doing so demonstrates dramatic effects on the malignancy of these cells. The RNAi approach relies on an innate cellular response to combat viral infection. In this process, double stranded mRNAs are recognized and cleaved by the dicer RNase resulting in 21-23 nucleotide long stretches of RNAi. These RNAis are incorporated into and unwound by the RNA-inducing silencing complex (RISC). The single antisense strand then guides the RISC to mRNA containing the complementary sequence resulting in endonucleolytic cleavage of the mRNA, see Elbashir et al. (Nature 411; 494-498 (2001)). Hence, this technique provides a means for the targeting and degradation of MG20 mRNA in tumor cells in vivo.

Antisense nucleic acid molecules of the invention can be designed using the nucleotide sequences of SEQ ID NO: 1 and/or the complementary strand of SEQ ID NO: 1 and/or the portions thereof, and constructed using enzymatic ligation reactions using procedures known in this art of the genetic engineering. For example, an antisense nucleic acid molecule (e.g., an antisense oligonucleotide) can be chemically synthesized using naturally occurring nucleotides or variously modified nucleotides designed to hybridize with a control region of a gene (e.g., promoter, enhancer, or transcription initiation region) to inhibit or control the expression of the MG20 gene through triple-helix formation. Alternatively, the antisense nucleic acid molecule may be designed to hybridize with the transcript of a gene (i.e., mRNA), and thus block the translation by inhibiting the binding of the transcript to ribosomes.

Like antisense, another type of gene therapy is designed as ribozymes, which aim to turn off a specific gene (e.g., MG20 gene) in a cell by targeting the mRNA transcripts copied from the gene. Ribozymes are RNA molecules that act as enzymes. Most often, they act as molecular scissors that cut RNA. The mechanisms of the action of ribozymes involve: delivery of RNA strands engineered to function as ribozymes; specific binding of the ribozyme RNA to mRNA encoded by the MG20 gene, for example; and cleavage of the target mRNA, preventing it from being translated into a protein, whereby the production of MG20 polypeptides/protein can be prevented

According to the invention there are provided nucleic acid fragments which are used as probes or primers in assays such as those described herein. “Probes” or “primers” are oligonucleotide sequences that hybridize in a base-specific manner to a complementary strand of nucleic acid molecules. As used herein, the term “primer” in particular refers to a single-stranded oligonucleotide which acts as a point of initiation of template-directed DNA synthesis using well-known methods (e.g., PCR). Such probes and primers include polypeptide-nucleic acids (PNAs), as described in Nielsen et al., Science 254:1497 (1991). PNA is a DNA mimic in which the nucleobases are attached to a pseudopeptide backbone (Good and Nielsen, Antisense Nucleic Acid Drug Dev. 7(4) 431-437, (1997) ). PNA is able to be utilized in numerous methods that traditionally have used RNA or DNA. In some cases PNA sequences perform better in techniques than the corresponding RNA or DNA sequences and have utilities that are not inherent to RNA or DNA. A review of PNA including methods of making, characteristics of, and methods of using, is provided by Corey (Trends Biotechnol. 15(6) :224-229 (1997)).

Typically, a probe or primer comprises a region of nucleotide sequences that hybridizes to at least about 15, preferably about 20-30, and more preferably about 40, 75, or 100, consecutive nucleotides of a nucleic acid molecule comprising a contiguous nucleotide sequence selected from SEQ ID NO: 1 or the complementary strand of SEQ ID NO: 1. In specific embodiments, a probe or primer comprises 100 or fewer nucleotides, preferably from 6 to 50 nucleotides, and more preferably from 12 to 30 nucleotides. In other preferred embodiments, the probe or primer is at least 70% identical, preferably at least 80% identical, more preferably at least 90% identical, or even more preferably at least 95% identical, to the contiguous nucleotide sequence or to the complementary strand of the contiguous nucleotide sequence. Generally, the probe or primer further comprises a label, e.g., radioisotope, fluorophore, enzyme, or enzyme cofactors (see, for example, Sambrook et al., Molecular Cloning: A Laboratory Manual, Cold Spring Harbor Laboratories, Cold Spring Harbor, N.Y., 1989).

The nucleic acid molecules of the invention can be identified and isolated using standard molecular biology techniques and the sequence information provided in SEQ ID NO: 1. For example, nucleic acid molecules can be amplified and isolated by PCR using synthetic oligonucleotide primers designed based on one or more of the sequences provided in SEQ ID NO: 1 and/or the complementary strand of SEQ ID NO: 1. Examples of the primers are, for example, set forth in SEQ ID NO: 5 and 6, respectively. See, for example, PCR (Eds. McPherson et al., IRL Press, Oxford) and U.S. Pat. No. 4,683,202. The nucleic acid molecules can be amplified using cDNA or mRNA as a template, cloned into an appropriate vector, and characterized by DNA sequence analysis. The amplified DNA can be radiolabeled and used as a probe for screening a cDNA library derived from human tissues, e.g., blood tissues, preferably a PBMC cDNA library.

One such amplification technique is inverse PCR (see Triglia et al., Nucl. Acids Res. 16:8186, 1988), which uses restriction enzymes to generate a fragment in the known region of the gene. The fragment is then circularized by ligation and used as a template for PCR with divergent primers derived from the known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or “RACE”, which identifies sequences that are 5′ and 3′ of a known sequence, see Frohman et al. (Meth. Enzymol. 218 : 340-356 (1993)).

A “variant” of the polynucleotide sequences as used herein includes a substantially homologous polynucleotide that is deviating in some bases from the identified polynucleotide, usually caused by mutations such as substitution, insertion, deletion or transposition. Polynucleotide variants preferably exhibit at least about 60%, more preferably at least about 70%, 80% or 90%, and even more preferably at least about 95%, 98% or 99% identity to the identified polynucleotide.

Portions or fragments or variants of the nucleotide sequences identified herein (and the corresponding complete gene sequences) can be used in various ways as polynucleotide reagents. For example, these sequences can be used to identify and express recombinant polypeptides for analysis, characterization, or therapeutic use. The sequences can additionally be used as reagents in the screening and/or diagnostic assays described hereinafter, and can also be included as components of kits (e.g., diagnostic kits) for use in the screening and/or diagnostic assays.

The invention further relates to nucleic acid constructs comprising a nucleic acid molecule selected from the group consisting of SEQ ID NO: 1 and the complementary strand of SEQ ID NO: 1 (or a portion, fragment or variant thereof). Such sequences can be suitably cloned into an expression vector via recombinant DNA techniques known widely in the art, to enable production of MG20 polypeptides. The term “expression vector” is used to denote a DNA molecule that is either linear or circular, into which another DNA sequence fragment of appropriate size can be integrated. Such DNA fragment(s) can include additional segments that provide for transcription of a gene encoded by the DNA sequence fragment. The additional segments can include and are not limited to regulatory sequences selected from: promoters, transcription terminators, enhancers, internal ribosome entry sites, untranslated regions, polyadenylation signals, selectable markers, origins of replication and such like. Expression vectors are often derived from plasmids, cosmids, viral vectors and yeast artificial chromosomes; vectors are often recombinant molecules containing DNA sequences from several sources. The vector thus includes one or more regulatory element(s), selected on the basis of the host cells to be used for expression, which is operably linked to the nucleic acid sequence to be expressed. Within the expression vector, “operably linked” is intended to mean that the nucleotide sequence of interest is linked to the regulatory element(s) in a manner which allows for expression of the nucleotide sequence.

Further, the invention is directed to host cells into which a recombinant expression vector of the invention has been introduced. The term “host cell” is understood to refer not only to the particular subject cell but also to the progeny or potential progeny of the foregoing cell. A host cell can be any prokaryotic (e.g., E. coli) or eukaryotic cell (e.g., yeast, insect cells, or mammalian cells, such as CHO or COS cells). Other suitable host cells are known to those skilled in the art.

Vectors can be introduced into prokaryotic or eukaryotic cells via conventional transfection or transformation techniques (see, Sambrook et al., supra).

The isolated nucleic acid molecules of the present invention may be formulated so as to permit entry into a mammalian cell and expression therein. Such vector formulations are particularly useful for therapeutic and/or screening purposes, as explained below. For example, an isolated nucleic acid molecule may be incorporated into a viral vector such as, but not limited to, retrovirus, adenovirus, or pox virus. Other formulations for transfection or therapeutic purposes include liposomes (i.e., artificial membrane vesicles), lipid-based systems, or microparticles (e.g. poly-lactide-co-glycolide microcapsules).

The present invention also relates to isolated polypeptides encoded by MG20 gene, and the immunogenic portions, fragments, derivatives and variants thereof. The term “polypeptide” refers to a polymer of amino acids, and not to a specific length; thus, peptides, oligopeptides, and proteins are included within the definition of polypeptide, and such terms may be used interchangeably herein unless specifically indicated otherwise. Particular polypeptides of interest in the context of this invention are amino acid subsequences comprising epitopes, i.e., antigenic determinants substantially responsible for the immunogenic properties of a polypeptide and being capable of evoking an immune response. Functional domains of the MG20 polypeptide are also considered to fall within the scope of the invention. Polypeptides also undergo maturation or post-translational modification processes that may include, but are not limited to: glycosylation, proteolytic cleavage, lipidization, signal peptide cleavage, propeptide cleavage, phosphorylation, and such like.

The term “isolated”, when applied to a polypeptide is a polypeptide that has been removed from its natural organism of origin. It is preferred that the isolated polypeptide is substantially free of other polypeptides native to the proteome of the originating organism. It is most preferred that the isolated polypeptide be in a form that is at least 95% pure, more preferably greater than 99% pure. In the present context, the term “isolated” is intended to include the same polypeptide in alternative physical forms whether it is in the native form, denatured form, dimeric/multimeric, glycosylated, crystallised, or in derivatized forms.

An “immunogenic portion” as used herein refers to a portion of a protein that is recognized by a T-cell and/or B-cell surface antigen receptor. The immunogenic portion generally comprises at least 5 amino acid residues, preferably at least 10, more preferably at least 20, and still more preferably at least 30 amino acid residues of a MG20 polypeptide or a variant thereof. Preferred immunogenic portions may contain a small N- and/or C-terminal fragment (e.g., 5-30 amino acids, preferably 10-25 amino acids).

A polypeptide “fragment” as used herein refers to a polypeptide derived from MG20 polypeptide, and comprises at least 6 contiguous amino acids. Useful fragments include those retain one or more of the biological activities of the polypeptide (e.g., fragments which are, for example, 6, 10, 15, 20, 25, 30, 40, 50, 100, or more amino acid in length). Biologically active fragments generally comprise a motif, domain, or segment that has been identified by analysis of the polypeptide sequence using well-known methods, e.g., signal peptides, extracellular domains, transmembrane segments, ligand binding regions, zinc finger domains, or glycosylation sites.

A polypeptide “variant” as used herein includes a substantially homologous polypeptide encoded by the same genetic locus. Polypeptide variants preferably exhibit at least about 70%, more preferably at least about 90%, and even more preferably at least about 95% identity to the identified polypeptide.

A chimeric or fusion polypeptide can be produced by standard recombinant DNA methods (see, for example, Ausubel, F. M. et al., supra). A fusion partner may assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the polypeptide or to enable the polypeptide to be targeted to desired intracellular compartments. Still further fusion partners include affinity tags, which facilitate purification of the polypeptide.

The present invention further provides antibodies and antigen-binding fragments thereof, that exhibit immunological binding to a polypeptide disclosed herein, or to a portion, fragment, or variant thereof. An antibody, or antigen-binding fragment thereof, is said to “specifically bind, “immunologically bind,” and/or is “immunologically reactive” to a polypeptide of the invention if it reacts at a detectable level with the polypeptide, and does not react detectably with unrelated polypeptides under similar conditions. Antibodies may be further capable of differentiating between patients with and without a neoplasm using the representative assays provided herein.

The term “antibody” as used herein refers to immunoglobulin molecules and immunologically active portion of immunoglobulin molecules. Antibodies may be prepared by any of a variety of techniques known to those skilled in the art (see, for example, Harlow and Lane, Antibodies: A Laboratory Manual, Cold Spring Harbor Laboratories, Cold Spring Harbor, N.Y., 1988). The present invention provides polyclonal and monoclonal antibodies that bind specifically to a polypeptide of the invention.

The term “monoclonal antibody” as used herein refers to a population of antibody molecules that contain only one species of antigen binding site capable of immunoreacting with a particular epitope of a polypeptide of the invention. Monoclonal antibodies of the invention may be coupled to one or more therapeutic agents. Suitable agents in this regard include differentiation inducers, drugs, toxins, and derivatives thereof. A therapeutic agent may be coupled (e.g., covalently bonded) to a suitable monoclonal antibody either directly or indirectly (e.g., via a linker group).

In addition, recombinant antibodies, such as chimeric and humanized antibodies, comprising both human and non-human portions, which can be made using standard recombinant DNA techniques, are within the scope of the invention. Also included within the term “antibody” are fragments, such as the Fab, F(ab′).

In general, antibodies of the invention can be used to detect the polypeptide (e.g., in a biological sample, such as human tissues, preferably PBMC) to evaluate the expression level of the polypeptide. Antibodies can be used prognostically or diagnostically to monitor MG20 protein levels in tissues as part of a clinical testing procedure.

The present invention is further directed towards the detection of MG20 expression in patients as a diagnostic tool. Hence, diagnostic assays for assessing MG20 gene overexpression or aberrant expression when compared to normal levels of MG20 expression are predictive of cancer in a patient. In one embodiment, the assays are used in the context of a biological sample (e.g., human tissue, blood, PBMC, etc.) to determine whether the subject is afflicted with neoplasms, or is at risk for developing neoplasms. The present invention also provides for predictive assays with means to determine whether the subject is susceptible to developing neoplasms. Such assays can be used for predictive purposes to thereby prophylactically treat a subject in need prior to the progression or metastasis of the neoplastic cells.

The term “normal level” when in the context of levels of gene expression, particularly MG20 gene expression, is used herein to denote the level of gene expression in healthy non-diseased tissue. Normal levels of expression represent the baseline or control level of expression of a gene. Aberrant expression levels in cells, either at levels that are too high or too low, are considered not to be normal and can be indicative of disease in the tissue from which the cells have been obtained, e.g. cancer.

The present invention further identifies particular cancers in which MG20 expression is elevated. Without limiting the invention to diagnosis and treatment of these particular cancers, MG20 elevated expression is seen in gastric, thyroid, uterine, ovarian, testis, kidney and prostate cancer.

The nucleic acids, probes, primers, polypeptides, and antibodies described herein can be used in diagnostic assays for the susceptibility to and/or presence of neoplastic diseases, such as cancer. In one embodiment, the present invention provides a novel and effective method for detecting neoplasm in a subject, which typically comprises detecting the level of MG20 or a portion, fragment or variant thereof in a biological sample; and comparing the level of MG20 or a portion, fragment or variant thereof in the biological sample with that in a control sample. An increased level of MG20 or a portion, fragment or variant thereof in the biological sample compared to that in the control sample, according to the invention, indicates the presence of neoplasm in the subject from which the biological sample was obtained.

As discussed in more detail below, in one embodiment of the invention the biological sample comprises PBMCs isolated from blood taken from a patient. Total mRNA is isolated from the PBMCs and analysed for the level of expression of MG20. Higher than normal levels of MG20 mRNA in the PBMCs is indicative of the presence of cancer in the patient. A particularly striking correlation has been observed between MG20 over-expression in PBMCs and the presence of gastric cancer in a patient.

As used herein, a “biological sample” refers to a cell or a population of cells or a quantity of tissues or body fluid, such as whole blood, serum, plasma, saliva, cerebrospinal fluid or urine from a subject or patient, wherein a quantity of tissues, e.g., blood tissues, removed from a human is more preferable. In contrast, a “control sample”, as used herein, refers to a sample that corresponds to the biological sample described above, but which demonstrates normal levels of MG20 expression (i.e., not affected by neoplasms). The level or amount of MG20 can be measured based on quantitative or qualitative methods, as described in detail below.

In a first method of detecting or diagnosing a susceptibility to and/or presence of neoplasms, hybridization methods, such as Southern analysis, Northern analysis, or in situ hybridization, can be used. The overexpression of MG20 gene can be indicated by hybridization of the gene in mRNA or cDNA to a nucleic acid probe as described above.

In a preferred embodiment, the assay employs RT-PCR, in which PCR is applied in conjunction with reverse transcription. Typically, RNA is extracted from a biological sample, and is reversely transcribed to produce cDNA molecules. PCR amplification using at least one specific primer generates a cDNA molecule, which may be separated and visualized using, for example, gel electrophoresis. Amplification may be performed on biological samples taken from a subject and on control samples taken from a healthy individual who is not afflicted with a neoplasm. The amplification reaction may be performed on several dilutions of cDNA spanning two orders of magnitude.

In yet another embodiment, assays of nucleic acid (e.g., oligonucleotide) probes that are complementary to target nucleic acid sequence segments from a subject, can be used to identify the expression of MG20 genes. In one embodiment, an oligonucleotide array (microarray) can be used. Oligonucleotide arrays generally comprise a plurality of different oligonucleotide probes that are immobilized on a surface of a substrate in different known spots. The surface is typically biocompatible. These oligonucleotide arrays, also called “DNA chips” or “biochips”, have been generally disclosed in the art, for example, U.S. Pat. Nos. 6,605,363, 6,528,291, 6,403,368, and 6,350,620.

In another embodiment of the invention, diagnosis of a susceptibility to and/or presence of neoplasms can also be made by examining expressions of the MG20 polypeptide in biological samples by a variety of methods, including, but not limited to, enzyme linked immunosorbent assays (ELISA), Western blots, immunohistochemical staining, radioimmunoassay, and immunoprecipitations.

Western blotting analysis, using an antibody as described above that specifically binds to a polypeptide encoded by MG20 gene, or the fragments, portions thereof, can be used to identify the difference between the expression level of MG20 genes in a biological sample and a control sample.

In accordance with one embodiment of the invention, the level or amount of MG20 gene expression, either in polynucleotides (e.g., mRNA or cDNA) or in polypeptides manner, in the biological sample that is higher than the normal level or amount in the control sample, is indicative of an elevated expression of MG20, and is diagnostic of a susceptibility to and/or presence of neoplasms.

Kits useful in the methods of diagnosis or detection comprise components useful in any of the methods as described herein, including for example, hybridization probes, amplification primers, or antibodies that bind specifically to MG20 polypeptides. Further, DNA chips or protein chips on which biomolecules such as oligonucleotides or oligopeptides can interact with MG20 gene or polypeptide, are also within the scope of the invention.

It will be appreciated by the skilled person that by providing means for detecting and monitoring expression and/or activity of MG20 in a patient, the invention provides valuable diagnostic and prognostic tools. It has become increasingly apparent that cancer is often a multifactorial disease where a more holistic approach to patient treatment and conservation is required. The invention, thus, provides a further effective means for attaining this goal. In cases where tumor management is a more appropriate regime of therapy than pursuing an outright cure, the diagnostic methods and kits of the invention allow for monitoring of tumor progression. In turn, the likely course of the disease (i.e. prognosis) and the nature of the cancer can be determined by monitoring MG20 expression and/or activity in the patient. Of course, MG20 levels can be monitored in isolation or in combination with other suitable markers of neoplasia.

In other embodiments, the present invention relates to the formulation of one or more of the polynucleotide, polypeptide, T-cell and/or antibody compositions described herein in pharmaceutically acceptable excipients or carriers for administration to a subject in need, either alone, or in combination with one or more other therapies.

It will also be understood that, if desired, the nucleic acid segment, RNA, DNA or PNA compositions that express a polypeptide as disclosed herein may be administered in combination with other agents as well, such as, e.g., other proteins or polypeptides or various pharmaceutically active agents. In fact, there is virtually no limit to other components that may also be included, given that the additional agents do not cause a significant adverse effect upon contact with the target cells or host tissues. The compositions may thus be delivered along with various other agents as required in the particular instance.

The formulation of pharmaceutically acceptable excipients and carriers is well-known to those skilled in the art, as is the development of suitable dosing and treatment regimens for using the particular compositions described herein in a variety of treatment regimens, including e.g., oral, parenteral, intravenous, intranasal, and intramuscular administration and formulation. Suitable pharmaceutical preparations may be in the form of tablets, pills, lotions, gels, liquids, powders, suppositories, suspensions, liposomes, microparticles or other appropriate formulations known in the art.

Vaccines that contain one or more of the MG20 polynucleotide, polypeptide, T-cell and/or antibody compositions described herein in combination with adjuvants, and that act for the purposes of prophylactic or therapeutic use, are also within the scope of the invention. The preparation of vaccines is generally described in, for example, M. F. Powell and M. J. Newman, eds., “Vaccine Design (the subunit and adjuvant approach)”, Plenum Press (NY, 1995). MG20 polypeptide fragments or peptides may comprise an immunogenic epitope, which can be identified using standard methods (Geysen et al. Proc. Natl. Acad. Sci. USA 81: 3998 (1983)). Such epitope bearing peptides typically contain at least ten to fourteen amino acid residues of SEQ ID NO: 2, and can be produced by fragmenting the MG20 polypeptide.

The invention also provides methods of treatment (prophylactic and/or therapeutic) for cancers using a therapeutic agent comprising polynucleotide, polypeptide, T-cell and/or antibody described herein.

In one embodiment, the method for treating cancers is an immunotherapy. The term “immunotherapy” as used herein may be active or passive immunotherapy. In the active immunotherapy, the treatment relies on the in vivo stimulation of the endogenous host immune system to react against tumors with the administration of immune response-modifying therapeutic agents (such as polypeptides and polynucleotides as provided herein). On the other hand, the treatment in the passive immunotherapy involves the delivery of therapeutic agents with established tumor-immune reactivity (such as effector cells or antibodies) that can directly or indirectly mediate antitumor effects and does not necessarily depend on an intact host immune system. Examples of effector cells include T-cells, T lymphocytes (such as CD8⁺ cytotoxic T lymphocytes and CD4⁺ T-helper tumor-infiltrating lymphocytes), killer cells (such as Natural Killer cells and lymphokine-activated killer cells), B cells and antigen-presenting cells (such as dendritic cells and macrophages) expressing a polypeptide provided herein.

In another embodiment, the method for treating cancers is a gene therapy. As used herein, “gene therapy” refers to administration of a therapeutic agent comprising polynucleotide sequences as described herein so that the expression of MG20 can be altered (i.e., inhibited or blocked).

In one preferred embodiment, an antisense construct of the invention can be used and delivered as an expression plasmid. When the plasmid is transcribed in the cell, it produces RNA that is complementary to a portion of the mRNA and/or DNA which encodes MG20 polypeptide. To perform antisense therapy, for example, oligonucleotides (mRNA, cDNA, or DNA) are designed that are complementary to the MG20 gene or the mRNA thereof, or as ribozymes. The antisense molecules bind specifically to MG20 genes or its mRNA transcripts and thus prevent transcription and/or translation. As described previously, alternative nucleotide based thearapies can also be employed, such as those based upon RNAi techniques.

The therapeutic agent(s) can be used concurrently, and are administered in a therapeutically effective amount (i.e., an amount that is sufficient to treat cancers, such as by ameliorating symptoms associated with cancers (e.g., cachexia), preventing or delaying the progression and/or development of cancers). The therapeutically effective amount will depend upon the symptoms and severity of the cancers, and can be determined by clinical physicians.

The invention also pertains to platforms and methods for identifying agents (e.g., prodrugs, antagonists, inhibitors, receptors, ligands, chemicals, herbal medicines, fusion proteins, peptidomimetics, binding agents, antibodies, ribozymes, or other drugs) that alter (e.g., decrease, increase, agonise, antagonise, moderate, inhibit, or block) the expression of MG20, interact with MG20 polynucleotide or MG20 polypeptide described herein, or inhibit the biological activity of MG20 in vivo.

In one embodiment, the invention provides platforms and assays for screening candidates that bind specifically to the polynucleotide or polypeptide of the invention, or modulate the expression of polypeptide or the fragments, portions thereof as described herein. Such candidates can be obtained using any of the numerous approaches in combinatorial library methods known in the art, including but not limited to, spatially addressable parallel solid phase libraries, synthetic libraries using affinity chromatography selection. All polypeptide, non-peptide oligomer, or small molecule libraries of compounds can be subjected to high-throughput screening using these methods.

MG20 protein-protein interactions or protein-small molecule interactions can be further investigated using technologies such as a BIAcore® which detects molecular interactions using surface plasmon resonance (BIAcore, Inc., Piscataway, N.J.; see also www.biacore.com).

In the above assays or platforms of the invention, it may be desirable to immobilize MG20 genes (e.g., full length or partial sequence set forth in SEQ ID NO: 1) or the polypeptide or the fragments, portions thereof, on a surface or solid support, to facilitate separation of the desirable agents as described above from a large of libraries.

Via the screening processes of the invention it is possible to identify novel agents that show an activity that alters MG20 biological function. Preferably, the agents identified in this manner are those inhibit or block the expression of MG20 genes, or those inhibit the biological activity of MG20. Hence, the invention provides methods for identifying MG20 interacting molecules typically via detection of a positive binding interaction between MG20 and a target molecule. Further screening steps may be used to determine whether the identified positive binding interaction is of pharmacological importance—i.e. whether the target molecule is capable of moderating MG20 biological activity or function.

If a molecule with a positive MG20 moderating effect is identified by the screening processes of the invention, the molecule is classified as a ‘hit’ and can then be assessed as a potential candidate drug. Additional factors may be taken into consideration at this time or before, such as the absorption, distribution, metabolism and excretion (ADME), bio-availability and toxicity profiles of the molecule, for example. If the potential drug molecule satisfies the pharmacological requirements it is deemed to be pharmaceutically compatible. Suitable compositions can be formulated for testing the activity in-vitro and in-vivo in accordance with standard procedures known in the art. Accordingly, it is within the scope of the invention to further use the candidate drug identified above in an appropriate animal model, in order to further determine the efficacy, toxicity, or side effects of treatment with such agents. Also, the agent(s) identified above can be used in an animal model to determine the mechanism of action of such agents. The potentially valuable molecules or prodrugs for the prevention and/or treatment of cancers and/or other diseases or disorders can thus be screened in the platforms and assays as described above.

The following is a detailed description of the examples of carrying out the inventions. This description is not to be taken in a limiting sense, but is made merely for the purpose of further illustrating the present inventions.

### EXAMPLES

### Example 1

### RT-PCR of MG20 in Peripheral blood Mononuclear Cells from a Healthy Volunteer and Cancer Cell Lines

I. Materials and Methods

A. Blood Sample:

Peripheral venous blood collected freshly from a healthy volunteer with sodium heparin was used as control, and peripheral blood mononuclear cells (PBMCs) were isolated therefrom using Ficoll-Paque plus (Amersham Biosciences).

B. Human Cell Lines:

Human cell lines such as a gastric cancer cell line (KATO III), breast cancer cell lines (MDA-MB-435S, MCF 7), hepatoma cell lines (Hep3B, HepG2), a prostate cancer cell line (DU 145), an esophageal cancer cell line (CE 146T/VGH), kidney cell lines (293, 293T) and a lung cancer cell line (NCI-H146) were purchased from BCRC, Taiwan. The cell lines, KATOIII and NCI-H146, were maintained in RPMI 1640 (Life Technologies, Inc.) supplemented with 10% fetal calf serum. The 293, 293T, DU 145, MDA-MB-435S and MCF 7 cell lines were maintained in DMEM (Life Technologies, Inc.) supplemented with 10% fetal calf serum. The Hep3B, HepG2 and CE 146T/VGH cell lines were maintained in DMEM with NEAA (Life Technologies, Inc.) supplemented with 10% fetal calf serum.

C. RNA Preparation:

The single step RNA isolation method was performed for RNA preparation by using TRIzol Reagent. In brief, PBMC cells or cell lines were homogenized in a TRIzol Reagent (Invitrogen, Life Technologies, Inc.) and put at room temperature (RT) for 5 min. 200 μl chloroform was added and the mixture was vortexed for 15 sec, put at room temperature (RT) for 3 min. After centrifugation, the RNA in the upper aqueous phase was precipitated with an equal volume of isopropanol and incubated for longer than 40 min, washed with 75% ethanol and dried under vacuum. The RNA pellet was then resuspended in diethyl pyrocarbonate (DEPC)-treated water, and the final RNA concentration was determined spectrophotometrically by measuring the absorbance at 260 nm and 280 nm (GeneQuant pro RNA/DNA Calculator, Amersham Pharmacia Biotech, England).

D. Reverse Transcription-PCR analysis:

Total RNA (5 μg) was reverse-transcribed using M-MLV Reverse Transcriptase (Invitrogen, Life Technologies, Inc.). Samples were amplified with PCR in a final reaction volume of 25 μl containing 2.5 μl of 10 times buffer (Amersham Pharmacia Biotech.), 0.1 μl of 10 mM dNTPs, 10 pmoles of each primer and 0.5 units of Taq DNA Polymerase (Amersham Pharmacia Biotech.). To confirm the presence and integrity of the cDNA template, the housekeeping gene, GAPDH, was amplified for each sample using primers GAPDH-5 (5′-ACCACAGTCCATGCCATCAC-3′; SEQ ID NO: 3) and GAPDH-3 (5′-TCCACCACCCTGTTGCTGTA-3′; SEQ ID NO: 4). Conditions were as follows: an initial denaturation step for 5 minutes at 94° C., then 50 seconds at 94° C., 45 seconds at 55° C., and 1 min at 72° C. for 30 cycles, followed by an elongation step for 10 minutes at 72° C.

MG20 RT-PCR was performed using the following primers: primer F (5′-GTACTTTCCGTCACTCCAAC-3′; SEQ ID NO: 5) and primer R (5′-ACCCCAACACATCATCCTC-3′; SEQ ID NO: 6). The parameters were as follows: The first denaturation step was at 94° C. for 4 minutes, followed by 35 cycles of denaturation at 94° C. for 50 seconds; Primer annealing occurred at 55° C. for 45 seconds, and elongation at 72° C. for 1 min. The final elongation step was conducted at 72° C. for 10 minutes.

II. Results

MG20 mRNA expression was first analyzed in PBMC cells from healthy individuals and cancer cell lines using RT-PCR technique. The amplified PCR products were analyzed by electrophoresis, and the result was shown in FIGS. 2A and 2B, which represents the mRNA expression of MG20 and GAPDH, respectively. 12.5 μl of every RT-PCR product was loaded in each lane. In FIGS. 2A and 2B, the mRNA used for RT-PCR is extracted from samples: PBMC from a healthy individual (lane 1), a gastric cancer cell line (KATOIII, lane 2), breast cancer cell lines (MDA-MB-435S, lane 3; MCF 7, lane 6), hepatoma cell lines (Hep3B, lane 4; HepG2, lane 10), a prostate cancer cell line (DU 145, lane 5), an esophagus cancer cell line (CE 146T/VGH, lane 7), kidney cell lines (293, lane 8; 293T, lane 9) and a lung cancer cell line (NCI-H146, lane 11), and M denotes the molecular size marker.

FIG. 2A shows the MG20 RT-PCR product with the expected molecular weight of 271 bp. It is noted that in FIG. 2A, the mRNA expression level of MG20 is elevated in cancer cell lines (lanes 2-11) when compared to that in the PBMC from a healthy individual (lane1), while in contrast, FIG. 2B shows that mRNA expression level of GAPDH, a housekeeping gene, appears no significant difference in all tested samples.

### Example 2

### Northern Blot Analysis of MG20 in Peripheral Blood Mononuclear Cells and Cancer Cell Lines

I. Materials and Methods

A. Blood Sample:

PBMC from a healthy volunteer was prepared as previously described in example 1.

B. Cell Lines:

A gastric cancer cell line (KATO III), breast cancer cell lines (MDA-MB-435S, MCF 7), a hepatoma cell line (HepG2), a prostate cancer cell line (DU 145), an esophagus cancer cell line (CE 146T/VGH), a kidney cell line (293T), a lung cancer cell line (NCI-H23) and a lung cell line (MRC-5) were used. All cell lines were purchased from BCRC, Taiwan. The cell lines, KATO III and NCI-H23, were maintained in RPMI 1640 (Life Technologies, Inc.) supplemented with 10% fetal calf serum. The cell lines, 293T, DU 145, MDA-MB-435S, MCF 7 and MRC-5, were maintained in DMEM (Life Technologies, Inc.) supplemented with 10% fetal calf serum. The cell lines, HepG2 and CE 146T/VGH, were maintained in DMEM with NEAA (Life Technologies, Inc.) and supplemented with 10% fetal calf serum.

C. Northern Blot Analysis:

Total RNA derived from different cancer line and PBMC were isolated from different cell lines. The single step RNA isolation method was used for RNA preparation as previously described. 20 μg of total RNA from PBMC or cancer cell lines were separated by electrophoresis on 1% agarose gels containing formaldehyde and transferred to a nylon filter (Immobilon-NY+, Millipore corporation). Blots were prehybridized at 42° C. for 1 hour in an ExpressHyb™ Hybridization Solution (Clontech, Palo Alto, Calif.), and then hybridized at 60° C. for 24 hours with CCDNBP1 DNA probe, which were radiolabeled with [α-³²P]dCTP (3000 Ci/mL; New England Nuclear) with use of a random priming method (Rediprime random primer labelling kit, Amersham). Blots were washed and autoradiographed with x-ray film at −70° C.

II. Result

As shown in FIG. 3A, the expression of human MG20 in different cancer lines and PBMC are shown as follows: a Hep G2 cell line (lane 1), a KATO III cell line (lane 2), a MCF 7 cell line (lane 3), a MDA-MB-435S cell line (lane 4), a DU 145 cell line (lane 5), a CE 146T/VGH cell line (lane 6), a NCI-H23 cell line (lane 7), a 293T cell line (lane 8) and a PBMC sample from a healthy volunteer (lane 9). FIG. 3B shows the blot probed with β-actin as a control. 10 μg of the product from Northern blot was loaded in each lane. The size of the MG20 cDNA was estimated to be approximately 1.4 kb.

### Example 3

### Multi-Tissue Northern Blot Analysis of MG20 in Normal, Non-Cancerous Tissues and Tumor Tissues

I. Materials and Methods

A. Multi-Tissue Blots:

MG20 mRNA expression in human normal, non-cancerous tissues and tumor tissues were studied by Northern blotting. Two multi-tissue blots were obtained, comprising a total of 12 different normal, non-cancerous tissues and 8 different tumor tissues.

B. Northern Blot Analysis:

Multi-tissue blots were prehybridized at 42° C. for 1 hour in an ExpressHyb™ Hybridization solution (Clontech, Palo Alto, Calif.) and then hybridized at 60° C. for 24 hours with CCDNBP1 DNA probe, which were labeled with [α-³²P]dCTP (3000 Ci/mL; New England Nuclear) by use of a random priming method (Rediprime random primer labelling kit, Amersham). Blots were washed and autoradiographed with x-ray film at −70° C.

II. Result

To examine the expression of the MG20 gene in various normal, non-cancerous tissues, Northern blot analysis was conducted and the results were shown in FIG. 4A. One major transcript of approximately 1.4 kb was detected in all the human normal, non-cancerous tissues, including brain (lane 1), heart (lane 2), skeletal muscle (lane 3), colon (lane 4), thymus (lane 5), spleen (lane 6), kidney (lane 7), liver (lane 8), small intestine (lane 9), placenta (lane 10), lung (lane 11), and peripheral blood leukocytes (lane 12). FIG. 4B shows the blot probed with β-actin which is of comparable intensity for each corresponding lane in FIG. 4A.

In FIG. 4A, a higher level of MG20 mRNA expression was observed in heart (lane 2), skeletal muscle (lane 3) and placenta (lane 10). when compared with other tissues, suggesting a potential negative role of MG20 in cell differentiation and proliferation.

Previous study has shown the relative low expression of MG20 in bone marrow among various immune tissues examined by RT-PCR. To examine the expression of the MG20 gene in various tumor tissues and its potential of being a cancer marker, Northern blot analysis of mRNA isolated from 8 different human tumor tissues, including breast tumor (lane 1), ovarian tumor (lane 2), uterine tumor (lane 3), lung tumor (lane 4), kidney tumor (lane 5), gastric tumor (lane 6), colon tumor (lane 7), and rectum tumor (lane 8) were to be tested.

The results are shown in FIG. 5A, wherein although one major MG20 transcript of approximately 1.4 kb was detected in all the human tumor tissues, it was relatively more highly expressed in human ovarian tumor (lane 2), uterine tumor (lane 3), kidney tumor (lane 5) and gastric tumor (lane 6) tissues than in other tumor tissues. FIG. 5B shows the blot probed with β-actin as a loading control, which is of comparable intensity for each corresponding lane in FIG. 5A.

### Example 4

### Detection of MG20 in Peripheral Blood Mononuclear Cells from Gastric Cancer Patients

In many countries, including Taiwan, America, Japan and China, gastric cancer has led to major mortality. In order to determine whether MG20 is a potential biomarker for gastric cancer, the expression of MG20 mRNA in peripheral blood mononuclear cells (PBMC) from both gastric cancer patients and healthy individuals was examined with RT-PCR techniques.

I. Materials and methods

A. Blood Sample:

Peripheral venous blood samples were collected from 13 patients with gastric cancer. Also, peripheral venous blood samples from 4 healthy volunteers were obtained to be used as controls. Patient samples were collected at the National Taiwan University Hospital, Taiwan. Peripheral blood mononuclear cells (PBMC) were isolated from freshly collected citrated venous blood using Ficoll-Paque (Amersham Biosciences).

B. Reverse Transcription-PCR analysis:

The single step RNA isolation method was used for RNA preparation as previously described, and RT-PCR analysis was performed as previously described in example 1.

II. Results

FIGS. 6A and 6B show the gene expression of MG20 and GAPDH, respectively. The mRNA samples were isolated from PBMC cells from healthy individuals (lanes 1-4) and gastric cancer patients (lanes 5-8), and the expression level of mRNA was analyzed by the RT-PCR technique. 12.5 μl of every RT-PCR product was loaded in each lane. As shown in FIG. 6A, a major band with the molecular weight being 271 bp was observed in these PBMC cell samples. It is noted that in FIG. 6A, the mRNA expression level of MG20 significantly increased by 2 folds on average in PBMC cells of gastric cancer patients (lane 5-8) when compared to that of healthy individuals (lane 1-4). In contrast, mRNA expression levels of GAPDH show no distinguishable differences in all tested samples.

### Example 5

### In vitro Transforming and in vivo Tumorigenic Activities of MG20

In order to determine whether MG20 is a potential oncogene, MG20 was transfected into NIH 3T3 cells and the changes of growth properties induced by MG20 overexpression were assayed by a serious of experiments.

I. Material and methods

A. Stable Transfection of MG20

NIH-3T3 cells were cultured in DMEM with 10% FBS at 37 C. The human MG20 coding sequence was cloned from KATO III cellular RNA with oligonucleotide primers. cDNA was synthesized by reverse transcriptase (Invitrogen) with Oligo dT primer and PCR amplified by Taq polymerase (Amersham Biosciences). The PCR product was then cloned into pGEM-T-Easy Vector (Promega). For transfection, MG20 was subcloned into pcDNA 3.1. This vector was introduced into NIH-3T3 cells by lipofectamine 2000 (Invitrogen, California, U.S.A.). The transfected cells were then cultured in complete medium containing 600 μg/mL geneticin (G418) for selecting of recombinant clones expressing G418 resistance. After 4 weeks, individual surviving clones in the presence of G418 were further expanded into a mass culture and the gene expression was examined by RT-PCR. The high expression clones were selected and subjected to transforming activity assays such as growth rate, focus formation, and anchorage independent growth in soft agar.

B. Cell Cycle Analysis

For cell cycle analysis, exponential growing cells (1×10⁶) were first trypsinized and fixed in 70% ethanol at −20° C. for 2 h, followed by treating with 10 μg/mL RNase A at 37° C. for 1 h and then stained with 50 μg/mL propidium iodide at room temperature in our dark room for 30 min. The stained cells were analyzed by flow-cytometer (PAS-II; Partec A G, Münster, Germany). Single-channel data were obtained and subsequently analyzed with a computer program (Phoenix Flow Systems, San Diego, Calif.), which is able to generate a plot of the number of cells versus DNA contents and the percentage of cells in cell cycle phases.

C. Anchorage Independent Growth in Soft Agar

For anchorage independent growth in soft agar, NIH-3T3 cells and MG20 transfectants were first suspended in a 2 ml 0.3% agar containing a complete medium and 20% FBS, and then were layered on a 1.5 ml solidified 0.6% agar in a complete medium and 20% FBS. Surviving colonies were developed with 2 mg/ml MTT solution at 37° C. for 12 h.

D. Tumorigenesis and Metastasis

Six to eight-week-old athymic nu/nu BALB/c mice obtained from National Laboratory Animal Center, Taiwan, were used in tumorigenic experiments. Animal care was provided in accordance with the procedures outlined in the Guide for the Care and Use of Laboratory Animals (NIH Publication No. 86-23, 1985). To assess tumorigenesis, parental and transfectant cells (2×10⁵) were suspended in 100 μl PBS and injected subcutaneously into posterior lateral aspect of the mice. Large (D) and small (d) diameters of growing tumors were measured twice a week, and the corresponding volumes (V) were estimated using the equation: V=width²×length×0.5. For the analysis of metastasis, 5×10⁶ cells in 100 μl were injected intravascularly into the tail vein of nude mice. Mice survival was followed and recorded. Surviving mice were killed after 5 weeks to examine metastatic nodules.

II. Results

A. In vitro Transforming Activity

To assess the transforming potential of MG20, the expression vectors containing MG20 coding sequence was transfected into NIH-3T3 cells. MG20 transfectants were obtained by G418 selection, and the expression level of MG20 was confirmed by RT-PCR analysis as shown in FIG. 7.

Three independent transfectants, MG 20-8, MG20-11 and MG20-12, were selected for transforming activity assays, including growth rate determination, morphology inspection, cell cycle progression examination, and anchorage independent growth in soft agar. As shown in FIG. 8, the transfectants exhibited morphologically transformed cell foci comprised of individual spindle-shaped cells characterized by a dense, disorganized pattern of growth, and increased refractivity (FIGS. 8C and 8D), while cells transfected with control vector retained a normal cell morphology (FIG. 8B). The tumorigenecity of MG-20 transfectants in vitro was also evaluated with clonogenic growth in soft agar before in vivo tests. This in vitro assay was known to be most closely related to in vivo tumorigenesis. MG20 transfected cells were able to form survival colonies in semisolid medium after MTT staining. TAB. 1 displays the proliferative properties of the transfected cell lines. A shorter doubling time and cell cycle progression from G1 to S phase were found in MG20 transfectants, but not in parental and vector control cells. The results in combination strongly suggest that MG20 is a oncogene that can transform NIH-3T3 cells.

B. In vivo Tumorigenic and Metastatic Activity

The tumorigenic ability of MG20 transfectants, vector control, or parental NIH-3T3 cells were tested in nude mice. The animals injected with MG20-12 cells rapidly developed tumors in 15 days in contrast to those with parental cells that had no evidence of tumor growth in the same period of time (FIG. 9). G20-12 transfectants had tumors reaching an average size of 2000 mm³. MG20-11 appeared less tumorigenic than other transfectants as only 3 out of 4 mice grew tumors after inoculation of 2×10⁶ cells (TAB. 1).

The metastatic potential of the MG20 transfectants are displayed in FIG. 10. Metastatic nodules, mostly in the intestinal and gastric walls, were found in the mice with MG20-tranfected cells, whereas no tumor was formed in the mice with parental cells or vector control cells even after 35 days. No death was found in mice with parental cells and vector control cells (FIG. 10 and TAB. 1).

### Example 6

### Clinical Relevance of MG20 Expression Level with Cancer

In order to determine the clinical relevance with cancer development, MG20 mRNA levels are measured in various kinds of cancer patients using RNA profiling assay.

I. Material and Methods

The cancer profiling array II which has been spotted with a variety of cancer tissue RNAs on a nylon membrane was purchased from Clontech, Palo Alto, Calif. The membrane contains a parallel array of normal and malignant RNAs of 19 different tissues including breast, ovarian, colon, stomach, lung, kidney, bladder, vulva, prostate, trachea, liver, uterus, cervix, rectum, thyroid gland, testis, skin, small intestine and pancreas. The RNAs of normal or malignant tissues were arranged in pairs, with each pair representing a particular type of normal and cancerous tissue from a single patient. For a single type of cancer, each line contains tissues from 3-10 patients. For the purpose of comparison, an additional 9 cell lines, including HeLa, Daudi, K562, HL60, G361, A549, MOLT4, SW480 and Raji were also spotted on the membrane. Profiling array was prehybridized at 68° C. for at least 30 mins in an ExpressHyb™ Hybridization Solution (Clontech, Palo Alto, Calif.), and then was further hybridized at 68° C. for 24 hours with MG20 DNA probes, which were radiolabeled with [α-³² P]dCTP 3000 Ci/mL (New England Nuclear) using of a random priming method (Rediprime random primer labelling kit, Amersham). Blots were washed and then autoradiographed with x-ray film at −70° C.

II. Results

FIG. 11A depicts the results of our study on the expression level of MG20 in various kinds of cancers. Significantly higher expression of MG20 was especially found in ovarian, thyroid, uterine, prostate and testis cancers, which, in turn, suggests that MG20 plays an important role in prostate, ovarian, uterine, testis and thyroid gland cancers.

### Example 7

### Knockdown of MG20 Expression Reduces Cell Malignancy

I. Materials and Methods

A. Antibody Production

For producing the antibody, GST fusion protein of MG20 was overexpressed as an antigen in E. Coli system and electrophoresed by SDS-PAGE. The purified GST-MG20 was contracted out to Cho Shui Shi Corp., Taiwan for antibody induction. Twenty mg of target protein and 2 ml of Freund's adjuvant was injected subcutaneously to a New Zealand white rabbit monthly. Blood samples were collected on a monthly basis after the second injection, and then the titer was analyzed. When the titer was acceptable, blood was sampled for serum collection. The contamination of antibody recognizing GST only was screened out using GST column and then the MG20 antibody was purified through MG20 affinity column.

B. RNAi

RNAi-mediated reduction in gene expression is performed by transfecting synthetic 19-21 nt double-stranded RNA. RNAi technology was used to explore the anti-tumor activity of these RNAis for MG20 through reducing the expression level of MG20. To obtain the best knock-down or silencing effect, the RNAi construct was obtained from Expression Arrest™ Human Short Hairpin RNA (shRNA) Libraries (Open Biosystems, U.S.A.).

C. Transfection of MG20-shRNA

DU-145 cells were cultured in DMEM with 10% FBS at 37° C. The MG20-shRNA and pSM2vector with irrelevant sequences were introduced into DU-145 cells by lipofectamine 2000 (Invitrogen, California, U.S.A.). The transfected cells were then cultured in complete medium containing 500 μg/mL puromycin for selecting of recombinant clones expressing puromycin resistance. After 5 days, all the transfectant cells were photographed and the gene expression was examined by RT-PCR.

II. Results

Interference of gene expression by small interfering RNA (siRNA or RNAi) is now recognized as a natural biological strategy for silencing gene expression. RNAi technology allows for gene-specific knock-down without induction of the non-specific interferon response in mammalian cell. We use RNAi targeting MG20 as an agent to reduce the expression level of MG20 and study the anti-malignancy effect of such agents.

A. RNAi Reduces Endogenous MG20 expression Levels in Cancer cells.

FIG. 12 shows the reduction of MG20 levels in MG20-shRNA transfectants. Using the amount of GAPDH as an internal control, it is obvious that the MG20 levels in shRNA containing cells is significantly lower than in cells without shRNA. Compared to the parental DU-145 cells, the relative amount of MG20 was 30% lower in DU-145 cells with MG20-shRNA constructs.

B. Cancer Cells with Reduced MG20 Level Exhibit Less Malignant Characteristics.

As shown in FIG. 13, cell proliferation of MG20-SI transfectants was significantly inhibited and cell morphology became normal-like (B and D) when compared with cells transfected with pSM2 vector with an irrelevant sequence (A and C) after 5 days. In addition, it appears that the MG20-shRNA construct exhibited the most significant influence on cell growth properties among these different designs. This result demonstrated the feasibility of using inhibitors of MG20 expression or function in treating malignancy in tumor cells.

Although particular embodiments of the invention have been disclosed herein in detail, this has been done by way of example and for the purposes of illustration only. The aforementioned embodiments are not intended to be limiting with respect to the scope of the appended claims, which follow. The choice of nucleic acid starting material, the clone of interest, or type of library used is believed to be a routine matter for the person of skill in the art with knowledge of the presently described embodiments. It is contemplated by the inventors that various substitutions, alterations, and modifications may be made to the invention without departing from the spirit and scope of the invention as defined by the claims.

1. A method of detecting and diagnosing cancer in a patient in need thereof, comprising the steps of obtaining a biological sample from said patient and analysing said biological sample for expression of MG20.

2. The method of claim 1, wherein expression of MG20 at a level in excess of a normal level is indicative of the presence of cancer.

3. The method of claim 1, wherein said sample comprises cells obtained from a biological source selected from the group consisting of: tissues; whole blood; serum; plasma; saliva; cerebrospinal fluid; ascites fluid; pleural fluid; and urine.

4. The method of claim 1, wherein said sample comprises cells obtained from a biopsy of a suspected tumor.

5. The method of claim 1, wherein said sample comprises peripheral blood mononuclear cells.

6. The method of claim 1, wherein said cancer is selected from the group consisting of: ovarian, thyroid, testis, uterine, prostate, kidney and gastric cancers.

7. A method of detecting and diagnosing cancer in a patient in need thereof, comprising the steps of:
(a) providing a biological sample from a subject;
(b) detecting the level of MG20 in the biological sample; and
(c) comparing the level of MG20, in the biological sample with that in a control sample obtained from a healthy individual;
wherein an increased level of MG20, in the biological sample compared to that in the control sample indicates the presence of neoplasm in the subject.

8. The method of claim 7, wherein said level of MG20 is the polynucleotide level or a portion, fragment, variant or complementary strand thereof, mRNA level, cDNA level, polypeptide level or a portion or fragment thereof, protein level or a level of biological activity of MG20.

9. The method according to claim 7, wherein said level of MG20 is detected by determining the mRNA level in said biological sample with the use of probe or primer that can hybridize with MG20 DNA, mRNA or a portion, fragment, variant or complementary strand thereof.

10. The method according to claim 9, wherein said primer comprises any one of SEQ ID NO: 5, SEQ ID NO: 6 or a combination thereof.

11. The method according to claim 1, wherein the expression of MG20 is analysed by determining the mRNA level of MG20, wherein the determination is detected by a technique selected from the group, without limiting, consisting of: Northern blot; in situ hybridization; RNase protection; RT-PCR based techniques; RACE; branched DNA technology; nucleic acid hybridization-based techniques, and combinations thereof.

12. The method according to claim 1, wherein the expression of MG20 is analysed by determining the polypeptide level of MG20 or a portion or fragment thereof.

13. The method according to claim 12, wherein said polypeptide level of MG20 is analysed by a technique selected from the group consisting of: Western blot; enzyme-linked immunosorbent assay (ELISA); radioimmunoassay; immunohistochemical staining; and combinations thereof.

14. The method according to claim 12, wherein said polypeptide level of MG20 is analysed by using a polyclonal or monoclonal antibody that binds to a MG20 polypeptide or a portion or fragment thereof.

15. A diagnostic kit suitable for detecting and diagnosing cancer in a patient in need thereof, comprising at least one nucleic acid probe that consists of a nucleotide sequence that is capable of hybridizing under conditions of medium stringency with about 15 contiguous bases of SEQ ID NO:1.

16. The kit of claim 15, wherein said nucleic acid probe is immobilized on a surface.

17. A diagnostic kit suitable for detecting and diagnosing cancer in a patient in need thereof, comprising at least one antibody, said antibody being capable of specifically binding to an MG20 polypeptide.

18. An in vitro method for monitoring the progression of cancer in a patient, comprising the steps of:
(a) contacting a biological sample from the patient with a substance selected from polynucleotides, probes, primers, or a portion, fragment, variant or complementary strand thereof; or polypeptides, antibodies that interact with MG20, or a portion or fragment thereof;
(b) detecting the level of MG20 polynucleotide or polypeptide, or the portion, fragment, variant or complementary strand thereof, that interacts with the substance in the biological sample;
(c) repeating steps (a) and (b) using a biological sample from the patient at a subsequent point of time; and
(d) comparing the level detected in step (c) with that in step (b) and therefrom monitoring the progression of the cancer in the patient.

19. The method of claim 18, comprising the further steps of obtaining a plurality of biological samples from said patient at a plurality of time intervals in a time course, and comparing the expression of MG20 in each sample, thereby effecting a diagnosis of cancer progression in said patient over said time course.

20. An in vitro method for monitoring the recurrence of cancer in a patient, comprising the steps of:
(a) interacting a biological sample from the patient following treatment with a substance selected from polynucleotides, probes, primers, or a portion, fragment, variant or complementary strand thereof; or polypeptides, antibodies that interact with MG20, or a portion or fragment thereof;
(b) detecting the level of MG20 polynucleotide or polypeptide, or the portion, fragment, variant or complementary strand thereof, that interacts with the substance in the biological sample;
(c) repeating steps (a) and (b) using a biological sample from the patient at a subsequent point of time; and
(d) comparing the level detected in step (c) with that from a control sample and therefrom determining the recurrence of the cancer in the patient.

21. An in vitro method for determining the prognosis of cancer in a patient, comprising the steps of:
(a) interacting a sample from the patient with a substance selected from polynucleotides, probes, primers, or a portion, fragment, variant or complementary strand thereof; or polypeptides, antibodies that interact with MG20, or a portion or fragment thereof;
(b) detecting the level of MG20 polynucleotide or polypeptide, or the portion, fragment, variant or complementary strand thereof, that interacts with the substance in the biological sample;
(c) comparing the level detected in step (b) with that from a control sample and therefrom determining the prognosis of cancer in the patient.

22. A polynucleotide vector comprising an isolated nucleic acid sequence that is substantially complimentary to at least 18 contiguous nucleotides of the nucleic acid molecule of SEQ ID NO:1, a transcription promoter, and a transcription terminator, wherein the promoter is operably linked to the nucleic acid sequence that is substantially complimentary to a nucleic acid molecule of SEQ ID NO:1, and wherein the isolated nucleic acid sequence that is substantially complimentary to a nucleic acid molecule of SEQ ID NO:1 is operably linked with the transcription terminator.

23. The polynucleotide vector of claim 22, wherein the vector is selected from the group consisting of a viral vector; and a plasmid vector.

24. The polynucleotide vector of claim 22, wherein the vector is an expression vector.

25. The polynucleotide vector of claim 22, wherein the vector is capable of generating RNAi in a transfected cell.

26. A mammalian cell comprising the polynucleotide vector of claim 22.

27. A recombinant host cell comprising the polynucleotide vector of claim 22, wherein the host cell is selected from the group consisting of bacterium; yeast; fungal cells; insect cells; mammalian cells; and plant cells.

28. A method of producing an MG20 polypeptide, the method comprising culturing recombinant host cells that comprise the polynucleotide vector of claim 22, and that produce said polypeptide, and isolating said polypeptide.

29. An antibody or antibody fragment that specifically binds to a polypeptide of SEQ ID NO: 2.

30. The antibody of claim 29, wherein the antibody is selected from the group consisting of: a polyclonal antibody; a murine monoclonal antibody; a humanized monoclonal antibody derived from a murine monoclonal antibody; a human monoclonal antibody; and a fab antibody fragment.

31. An anti-idiotypic antibody that specifically binds to an antibody or antibody fragment of claim 29.

32. A method for inhibiting malignancy in cancer cells comprising exposing said cancer cells to an inhibitor of MG20.

33. The method of claim 32, wherein said inhibitor of MG20 comprises a moiety selected from the group consisting of: a polynucleotide sequence that is substantially complimentary to the sequence of SEQ ID NO: 1; an oligonucleotide sequence that is substantially complimentary to at least 12 contiguous bases of SEQ ID NO:1; an oligonucleotide RNAi sequence that is substantially complimentary to at least 18 contiguous bases of SEQ ID NO:1; an antibody of claim 29; a small molecule; a glycoprotein; and a polysaccharide.

34. A pharmaceutical composition for prevention, treatment and/or therapy of cancer in a patient in need thereof, comprising:
an inhibitor of MG20 selected from the group consisting of: a polynucleotide sequence that is substantially complimentary to the sequence of SEQ ID NO: 1; an oligonucleotide sequence that is substantially complimentary to at least 12 contiguous bases of SEQ ID NO:1; an oligonucleotide RNAi sequence that is substantially complimentary to at least 18 contiguous bases of SEQ ID NO:1; an expression vector construct comprising said polynucleotide sequence; an expression vector construction comprising said oligonucleotide sequence; an antibody as described previously; a small molecule; a glycoprotein; and a polysaccharide; and
a pharmaceutically acceptable excipient and carrier.

35. A pharmaceutical composition for prevention, treatment and/or therapy of neoplasm, comprising:
a polypeptide with the sequence set forth in SEQ ID NO:2, or a portion or fragment thereof; and
a pharmaceutically acceptable excipient and carrier.

36. A method for prevention, treatment and/or therapy of cancer, and inhibition of the development of cancer in a patient in need thereof, comprising administering to the subject an effective amount of pharmaceutical composition according to claim 34.

37. A method for prevention, treatment and/or therapy of cancer, and inhibition of the development of cancer in a patient in need thereof, comprising administering to the subject an effective amount of pharmaceutical composition according to claim 35.

38. A pharmaceutical composition for prevention, treatment and/or therapy of cancer in a patient in need thereof, comprising:
a polynucleotide sequence with the sequence set forth in SEQ ID NO: 1; or a portion, fragment, variant or complementary strand thereof, an expression vector construct containing said polynucleotide or host cell transformed or transfected with said expression vector; and
a pharmaceutically acceptable excipient and carrier.

39. A method for prevention, treatment and/or therapy of cancer, and inhibition of the development of cancer in a patient in need thereof, comprising administering to the subject an effective amount of pharmaceutical composition according to claim 38.

40. A vaccine composition, comprising a polypeptide of SEQ ID NO:2, or an antigenic fragment of said polypeptide, and a pharmaceutically acceptable carrier.

41. The vaccine composition of claim 40, further comprising a non-specific immune response adjuvant.

42. A vaccine composition, comprising a polynucleotide vector of claim 22, and a pharmaceutically acceptable carrier.

43. The vaccine composition of claim 40, further comprising a non-specific immune response adjuvant.

44. A method for identifying a molecule that interacts with MG20 comprising:
a) screening a plurality of candidate molecules in order to identify one or more target molecules that bind to MG20 polypeptide;
b) determining whether said one or more target molecules interacts with MG20 polypeptide so as to moderate MG20 biological activity; and
c) characterising a target molecule that moderates MG20 biological activity as an MG20 interacting molecule.

45. The method of claim 44, wherein the MG20 interacting molecule is further characterised for pharmaceutical compatibility.

46. The method of claim 44, wherein the MG20 interacting molecule is an inhibitor of MG20 biological activity.

47. The method of claim 44, wherein the MG20 interacting molecule is an enhancer of MG20 biological activity.

48. The method of claim 44, wherein the MG20 interacting molecule is a protein.

49. The method of claim 44, wherein the MG20 interacting molecule is a peptide.

50. The method of claim 44, wherein the MG20 interacting molecule is a small molecule.

51. The method of claim 44, wherein the MG20 protein in part (a) is immobilised on a surface.

52. The method of claim 44, wherein the screening step of part (a) is performed using a BIAcore.

53. A method for identifying a molecule that moderates expression MG20 in a cell comprising:
a) exposing a cell that expresses MG20 to a candidate molecule in order to identify whether said candidate molecule has a moderating effect on expression of MG20 in the cell;
b) determining whether said candidate molecule moderates MG20 expression levels; and
c) characterising a candidate molecule that selectively moderates MG20 expression levels in the cell as an MG20 moderator molecule.

54. The method of claim 53, wherein the MG20 moderator molecule is further characterised for pharmaceutical compatibility.

55. The method of claim 53, wherein the MG20 moderator molecule is an inhibitor of MG20 expression.

56. The method of claim 53, wherein the MG20 moderator molecule comprises a nucleotide sequence that hybridizes with at least 18 contiguous nucleotides of SEQ ID NO:1.

57. A MG20 moderator molecule identified according to the method of claim 53.


SEQUENCE LISTING (6 sequences)

SEQ ID NO: 1 - DNA, 1540 bp, Homo sapiens
  CDS (75)..(1157)
ggagcggctgtcgcagtgcggctccggcagtggcagcggaggcctgtgtttgcggccttcggcaagcgactgagatggcgagcgcaactgcacctgcagccgcagtccccaccctggcttcgcctttggagcagctccggcacttggcggaggagctgcggttgctcctgcctcgagtgcgggtcggcgaagcccaggagaccaccgaggagtttaatcgagagatgttctggagaagactcaatgaggcagctgtgactgtgtcaagggaagccacgactctgaccatagtcttctctcagcttccactgccgtctccacaggaaacccagaagttctgtgaacaagtccatgctgccatcaaggcatttattgcagtgtactatttgcttccaaaggatcaggggatcaccctgagaaagctggtacggggcgccaccctggacatcgtggatggcatggctcagctcatggaagtactttccgtcactccaactcagagccctgagaacaatgaccttatttcctacaacagtgtctgggttgcgtgccagcagatgcctcagataccaagagataacaaagctgcagctcttttgatgctgaccaagaatgtggattttgtgaaggatgcacatgaagaaatggagcaggctgtggaagaatgtgacccttactctggcctcttgaatgatactgaggagaacaactctgacaaccacaatcatgaggatgatgtgttggggtttcccagcaatcaggacttgtattggtcagaggacgatcaagagctcataatcccatgccttgcgctggtgagagcatccaaagcctgcctgaagaaaattcggatgttagtggcagagaatgggaagaaggatcaggtggcacagctggatgacattgtggatatttctgatgaaatcagccctagtgtggatgatttggctctgagcatatatccacctatgtgtcacctgaccgtgcgaatcaattctgcgaaacttgtatctgttttaaagaaggcacttgaaattacaaaagcaagtcatgtgacccctcagccagaagatagttggatccctttacttattaatgccattgatcattgcatgaatagaatcaaggagctcactcagagtgaacttgaattatgacttttcaggctcatttgtactctcttcccctctcatcgtcatggtcaggctctgatacctgcttttaaaatggagctagaatgcttgctggattgaaagggagtgcctatctatatttagcaagagacactattaccaaagattgttggttaggccagattgacacctatttataaaccatatgcgtatatttttctgtgctatatatgaaaaataattgcatgatttctcattcctgagtcatttctcagagattcctaggaaagctgccttattctctttttgcagtaaagtatgttgttttcattgtaaagatgttgatggtctcaataaaatgctaacttgccagtgattaaaaaaaaaaaaaaaaaaaaaaaaaaaaa

SEQ ID NO: 2 - protein, 360 aa, Homo sapiens
MASATAPAAAVPTLASPLEQLRHLAEELRLLLPRVRVGEAQETTEEFNREMFWRRLNEAAVTVSREATTLTIVFSQLPLPSPQETQKFCEQVHAAIKAFIAVYYLLPKDQGITLRKLVRGATLDIVDGMAQLMEVLSVTPTQSPENNDLISYNSVWVACQQMPQIPRDNKAAALLMLTKNVDFVKDAHEEMEQAVEECDPYSGLLNDTEENNSDNHNHEDDVLGFPSNQDLYWSEDDQELIIPCLALVRASKACLKKIRMLVAENGKKDQVAQLDDIVDISDEISPSVDDLALSIYPPMCHLTVRINSAKLVSVLKKALEITKASHVTPQPEDSWIPLLINAIDHCMNRIKELTQSELEL

SEQ ID NO: 3 - DNA, 20 bp, Homo sapiens
accacagtccatgccatcac

SEQ ID NO: 4 - DNA, 20 bp, Homo sapiens
tccaccaccctgttgctgta

SEQ ID NO: 5 - DNA, 20 bp, Homo sapiens
gtactttccgtcactccaac

SEQ ID NO: 6 - DNA, 19 bp, Homo sapiens
accccaacacatcatcctc